(19) United States
(12) Patent Application Publication
(10) Pub. No.: US 2019/0209523 A1
(43) Pub. Date: Jul. 11, 2019

(54) NOOTROPIC AND SKIN SUPPLEMENT COMPOSITIONS AND METHOD TO PROVIDE A TAILORED SUPPLEMENT

(71) Applicant: Staterra Inc., Woodlawn (CA)

(72) Inventors: Alysen CLARK, Ottawa (CA); Sara MOROZ, Nepean (CA); Kevin CLARK, Woodlawn (CA)

(21) Appl. No.: 16/244,719

(22) Filed: Jan. 10, 2019

(30) Foreign Application Priority Data
Jan. 10, 2018 (CA) 2991525

(51) Int. Cl. A61K 31/375; A61K 31/145; A61K 31/198; A61K 36/15; A61K 36/31; A61K 31/205; A61K 31/7028; A61K 31/675; A61K 31/194; A61P 25/28; A61K 8/65; A61K 8/60; A61K 8/67; A61K 8/49; A61K 8/35; A61K 8/9767; A61K 8/44; A61Q 19/00

(52) U.S. Cl. CPC A61K 31/375; A61Q 19/00; A61K 31/198; A61K 36/15; A61K 36/31; A61K 31/205; A61K 31/7028; A61K 31/675; A61K 31/194; A61P 25/28; A61K 8/65; A61K 8/60; A61K 8/675; A61K 8/671; A61K 8/678; A61K 8/4986; A61K 8/355; A61K 8/9767; A61K 8/44; A61K 31/145

(57) ABSTRACT

The present disclosure provides at least one skin supplement composition that has been shown to be advantageous for healthy skin, preferably comprised of hydrolyzed collagen, glucosamine sulfate, biotin, vitamin A palmitate, vitamin E, alpha lipoic acid, coenzyme Q₁₀, pine bark extract, L-selenomethionone and niacinamide. An advantageous nootropic composition is also disclosed preferably comprised of ascorbic acid, taurine, elemental magnesium, glycinate, L-theanine, L-tyrosine, pine bark extract, N-acetyl-L-carnitine, DL-choline bitartrate, maca root, glucosinolates, P5P (pyridoxal-5-phosphate) and alpha ketoglutaric acid. A method to provide a specifically tailored supplement is also disclosed, preferably of collecting dry blood spots, analyzing the dry blood spots with tandem mass spectrometry, determining an amino acid kinetic pattern based on the analysis, and generating the specifically tailor supplement based on the pattern.

### FIELD OF THE INVENTION

The invention relates to the field of skin supplements and nootropics, and more specifically to nootropic and skin supplement compositions and methods therefor.

### BACKGROUND OF THE INVENTION

There are several skin and nootropic supplements presently out on the market. The present disclosure provides a multi-facetted approach when designing the skin and nootropic supplement. The present supplements contain several individual natural health ingredients that will work together and synergistically to provide their benefits. For the disclosed skin supplement, glucosamine and hydrolyzed collagen can work in a synergistic fashion to enhance HA production and endogenous collagen production, respectively. While glucosamine contributes to the hydration and elasticity of the skin through the biosynthesis of HA, hydrolyzed collagen boosts the elasticity and helps hydrate the skin by stimulating collagen production. Similarly, the disclosed nootropic supplement contains multiple ingredients, which will collectively provide cognitive stimulation, neurotransmitter and metabolic support, and an energy fuel source.

### SUMMARY OF THE INVENTION

The present disclosure provides a nootropic composition comprising ascorbic acid, taurine, elemental magnesium glycinate, L-theanine, L-tyrosine, pine bark extract, N-acetyl-L-carnitine, DL-choline bitartrate, maca root, glucosinolates, P5P (pyridoxal-5-phosphate) and alpha ketoglutaric acid.

In an aspect, the present disclosure provides a skin supplement composition comprising hydrolyzed collagen, glucosamine sulfate, biotin, vitamin A palmitate, vitamin E, alpha lipoic acid, coenzyme Q₁₀, pine bark extract, L-selenomethionone and niacinamide.

In another aspect, the present disclosure provides for a method to provide a specifically tailored supplement, the steps comprising collecting dry blood spots; analyzing the dry blood spots with tandem mass spectrometry; determining an amino acid kinetic pattern based on the analysis; and, generating the specifically tailor supplement based on the pattern.

### BRIEF DESCRIPTION OF THE DRAWINGS

The following FIGURE serves to illustrate various embodiments of features of the disclosure. This FIGURE is illustrative and not intended to be limiting.

FIG. 1 is a flow chart of a method to design a specifically tailored supplement in accordance with an embodiment of the present disclosure.

### DETAILED DESCRIPTION OF THE INVENTION

The following embodiments are merely illustrative and are not intended to be limiting. It will be appreciated that various modifications and/or alterations to the embodiments described herein may be made without departing from the disclosure and any modifications and/or alterations are within the scope of the contemplated disclosure.

The present disclosure describes at least one skin supplement composition that has been shown to be advantageous for healthy skin. The skin supplement composition is preferably comprised of at least hydrolyzed collagen, glucosamine sulfate 2KC1, biotin (100%), vitamin A palmitate (297,000 IU/g), vitamin E (acetate) (734 IU/g) (100 IU=136.24 mg), alpha lipoic acid 99% powder, CoEnzyme Q₁₀ 99%, pine bark extract, elemental selenium [L-selenomethionone (0.5%)] and niacinamide (100%). The chart below illustrates the preferred daily dose and the preferred range of each ingredient.

TABLE A

| | Daily | Min | Max | |
|---|---|---|---|---|
| Ingredient | Dose | Range | Range | UoM |
| Hydrolyzed Collagen | 1000 | 500 | 3000 | mg |
| Glucosamine Sulfate | 800 | 500 | 1500 | mg |
| 2KCI | | | | |
| Biotin (100%) | 0.1 | 0.05 | 0.2 | mg |
| Vitamin A Palmitate | 695 | 200 | 1000 | mcg |
| (297,000 IU/g) | | | | RAE |
| Vitamin E (Acetate) | 134 | 50 | 500 | mg α- |
| (734 IU/g) (100 IU = | | | | tocopherol |
| 136.24 mg) | | | | |
| Alpha Lipoic Acid | 200 | 50 | 400 | mg |
| 99% powder | | | | |
| Coenzyme Q₁₀ 99% | 50 | 30 | 100 | mg |
| Pine Bark Extract | 100 | 75 | 300 | mg |
| Elemental Selenium | 100 | 50 | 150 | mcg |
| [L-selenomethionone | | | | |
| (0.5%)] | | | | |
| Niacinamide (100%) | 20 | 15 | 150 | mg |

The present skin supplement composition preferably provides increased skin hydration, oxidation, clarification and improved skin pigmentation.

With specific reference to skin hydration, a worker skilled in the art would appreciate that the ability for skin to retain water is related to the stratum corneum (SC), which is the upper layer of the skin. Hyaluronic acid (HA) in the dermis, the innermost layer of the skin, is a high molecular weight glycosaminoglycan that has hydrophilic properties and contributes to the hydration and elasticity of the skin. Research demonstrates that HA is naturally present in the epidermis and also thought to play a role in SC hydration. Collagen, responsible for giving skin its elasticity, is also essential for healthy skin and is the primary component of connective tissue located in the dermis. Collagen is made by fibroblasts, which are skin cells that provide the skin its strength and elasticity. Research shows that exposure to environmental factors and ageing cause a decrease in the levels of collagen in the body, which leads to a loss of elasticity and hydration. Glucosamine, found to occur naturally in human tissues, stimulates the biosynthesis of glycolipids, glycoproteins, proteoglycan, glycosaminoglycan and HA. Glucosamine sulphate is produced from shellfish, and has been shown to help treat hyperpigmentation and provide moisture for the skin by stimulating the biosynthesis of HA. The production of HA contributes to skin hydration and elasticity as HA has a high water retaining ability. Hydrolyzed collagen is produced by breaking down collagen, found in the bones, skin and connective tissue of animals. During the hydrolysis process, peptides are generated having varying molecular weight. Hydrolyzed collagen contains 19 amino acids, where approximately 50% of the total amino acids are glycine, proline and hydroxyproline. Hydrolyzed collagen is deficient in isoleucine, threonine and methionine. Hydrolyzed collagen has been shown to be safe as an oral supplement (FDA) and beneficial effects have been observed in the skin. Hydrolyzed Collagen is thought to affect skin by increasing the density of collagen fibrils and fibroblasts, which stimulate collagen production. The increased density of fibroblasts provides the skin with its strength and elasticity.

In view of the aforementioned, the present skin supplement composition contains glucosamine and hydrolyzed collagen to enhance the ability of the skin to hold moisture. Indeed, it is hypothesized that glucosamine and hydrolyzed collagen work in a synergistic fashion to enhance HA production and endogenous collagen production, respectively. While glucosamine sulphate contributes to the hydration and elasticity of the skin through the biosynthesis of HA, hydrolyzed collagen boosts the elasticity and helps hydrate the skin by stimulating collagen production. HA is also needed to bind collagen with elastin, which is the fibre that gives skin its stretch. Together, collagen and elastin work by retaining water and improving the elasticity of the skin. Additionally, research has suggested that pine bark extract can up-regulate genes related to de novo collagen production and promote enhanced skin hydration.

With specific reference to skin oxidation, the present skin supplement composition contains a targeted antioxidant blend preferably comprised of vitamin E, alpha lipoic acid (ALA), coenzyme Q₁₀, selenium and pine bark extract. These ingredients serve to decrease oxidative stress within various tissues. The presence of free radicals in the skin results in fine lines and wrinkles, which can be a direct cause of sun damage and the aging process. Vitamin E is a fat-soluble antioxidant that reacts with dangerous reactive-lipid radicals formed in fatty (lipid) tissues (adipose tissue) and membranes. Vitamin E is a group of compounds that includes tocopherols and tocotrienols. Although -tocopherol is the most common form found in North American diets followed by α-tocopherol, α-tocopherol is the most abundant form in the plasma. As such, vitamin E present in the plasma can react with the reactive oxygen species (ROS) generated by exposure to an oxidative environment such as UV light exposure, ozone in the air or other chemical exposure. Vitamin E is also the primary antioxidant in the outermost skin layer. When the skin is exposed to an oxidative environment, vitamin E levels are rapidly depleted. Vitamin E becomes a free radical itself once it reacts with the ROS. ALA is a fatty acid present in the mitochondria and provides reduction of oxidation by increasing anti-oxidant enzymes. The present skin supplement composition includes ALA for its antioxidant property as well as its ability to indirectly recycle vitamin E. A worker skilled in the art would appreciate that ALA indirectly recycles vitamin E by regenerating glutathione, the primary protective antioxidant of the body, which in turn recycles vitamin C. As vitamin C is needed to recycle vitamin E, ALA creates a synergistic and desired antioxidant effect. The present skin supplement composition also preferably contains selenium, the mineral antioxidant, as selenium is a cofactor to the powerful antioxidant glutathione. Although it is known that the body naturally makes glutathione, supplemented glutathione is poorly absorbed. However, supplementation can help support endogenous glutathione and animal studies suggest that selenium could be an important dietary preventative in the formation of skin tumors. The present skin supplement composition also contains pine bark extract, also known as pycnogenol (PYC), as a source of procyanidins, a group of powerful antioxidants. Research shows that PYC doubles the intracellular synthesis of anti-oxidative enzymes and acts as a potent scavenger of free radicals. Furthermore, other PYC roles may be involved in the regeneration and protection of vitamin C and E. Procyanidins have been shown to decrease the minimal erythema dose in human studies, which suggests that procyanidins have the ability to protect skin from UV damage. The skin supplement composition further contains coenzyme Q₁₀ for its anti-oxidant properties and its ability to boost cellular energy, as it acts as a coenzyme to adenosine triphosphate. Coenzyme Q₁₀ is localized primarily to the SC and functions to protect the deeper layers from damage caused by UV A. Studies demonstrate a reduction of free radicals and an increase in antioxidant capacity. Decreasing oxidative damage has been shown to reduce the appearance of new fine lines and wrinkles, prevent and decrease sun damage and slow the aging process. The present skin supplement composition is novel, and superior to other compositions in the market in part due to the multifaceted approach to free radical scavenging described above.

With specific reference to skin pigmentation, the disclosed skin supplement composition is preferably comprised of glucosamine and niacinamide, which act to lighten the appearance of sun spots and even skin complexion. Melanocytes, the melanin-producing cells, are found in the bottom layer of the skin epidermis. The effective transfer of melanosomes, specialized pigment-producing cells, from melanocytes to keratinocyte is required for pigmentation. The skin benefits of niacinamide (vitamin B3), are well documented. In the skin, niacinamide works as a powerful antioxidant and has been shown to lighten skin pigmentation when applied topically by inhibiting the transfer of melanosome. Glucosamine has also been shown to work topically to lighten skin pigmentation. In vitro studies have shown that glucosamine inhibits the activation of tyrosinase, which is an enzyme required for melanin production. The skin supplement composition will preferably lighten hyperpigmented areas in a synergistic fashion, both by inhibiting melanin production and also preventing its translocation to the keratinocyte. In addition to the direct pigmentation role, glucosamine also acts in an anti-inflammatory and immunomodulatory fashion. This reduces skin inflammation, redness and swelling, which results in a smoother, more even skin complexion.

With specific reference to skin clarification, the disclosed skin supplement composition is preferably comprised of vitamin A palmitate, also known in the art as retinyl palmitate, and biotin, the former of which acts to promote growth of new skin cells and the latter of which protects the skin from the sun, pollution and chemical damage, thereby clarifying and strengthening the skin and adding elasticity. A worker skilled in the art would understand that retinoids are synthetic derivatives of vitamin A that have been used for acne in a topical or systematic form. Vitamin A palmitate is converted to retinol, a first generation retinoid, after it is absorbed in the skin. Retinoids are shown to combat acne in several ways. Research demonstrates that retinoids prevent dead skin cells from sloughing off and reducing the amount of oil that skin produces (both of which clog pores), suppress androgen formation (which is a major cause of acne) and protect fats form oxidation. Deficiency in biotin can cause several symptoms including seborrheic dermatitis and dry skin. Seborrheic dermatitis can raise the risk of getting other medical conditions, including but not limited to acne. It is suggested that the synergistic effects of vitamin A and biotin will leave the skin clear and acne free.

The present disclosure further describes at least one nootropic composition that has been shown to be advantageous over existing nootropics. The nootropic composition is preferably comprised of at least L-leucine, L-isoleucine, L-valine, Taurine, Elemental Magnesium, Elemental Potassium, Sea Salt, L-theanine, L-tyrosine, pyridoxal-5-phosphate, pine bark extract, N-acetyl-L-carnitine, alpha ketoglutaric acid, DL-choline bitartrate, and Maca Root. The chart below illustrates the preferred daily dose and the preferred range of each ingredient.

TABLE B

| | Daily | Min | Max | |
|---|---|---|---|---|
| Ingredient | Dose | Range | Range | UoM |
| L-Leucine | 500 | 150 | 1500 | mg |
| L-Isoleucine | 250 | 75 | 1000 | mg |
| L-Valine | 250 | 100 | 1000 | mg |
| Taurine | 1000 | 500 | 3000 | mg |
| Elemental Magnesium | 10 | 5 | 150 | mg |
| (Magnesium (20%) Glycinate | | | | |
| Elemental Potassium | 200 | 100 | 200 | mg |
| (Potassium (36%) Citrate) | | | | |
| Sea Salt | 50 | 20 | 200 | mg |
| L-Theanine | 200 | 130 | 350 | mg |
| L-Tyrosine | 5000 | 500 | 10000 | mg |
| Pine Bark Extract | 200 | 75 | 600 | mg |
| N-Acetyl-L-Carnitine | 3000 | 600 | 4000 | mg |
| DL- Choline Bitartrate (100%) | 500 | 30 | 1000 | mg |
| Maca Root PE 0.6% | 800 | 500 | 3000 | mg |
| Glucosinolates | | | | |
| P5P (Pyridoxal-5-Phosphate) | 25 | 20 | 50 | mg |
| (100%) | | | | |
| Alpha Ketoglutaric Acid | 500 | 300 | 1000 | mg |

The present disclosure further describes an alternative nootropic composition that has similarly been shown to be advantageous over existing nootropics, containing different ingredients in different dosages as described below. Indeed, the nootropic composition is preferably comprised of at least ascorbic acid, taurine, elemental magnesium, glycinate, L-theanine, L-tyrosine, pine bark extract, N-acetyl-L-carnitine, DL-choline bitartrate, maca root, glucosinolates, P5P (pyridoxal-5-phosphate) and alpha ketoglutaric acid. The chart below illustrates the preferred daily dose and the preferred range of each ingredient.

TABLE C

| | Daily | Min | Max | |
|---|---|---|---|---|
| Ingredient | Dose | Range | Range | UoM |
| Ascorbic Acid | 500 | 200 | 1000 | mg |
| Taurine | 1000 | 500 | 3000 | mg |
| Elemental Magnesium | 30 | 5 | 150 | mg |
| (Magnesium (20%) Glycinate | | | | |
| L-Theanine | 200 | 130 | 350 | mg |
| L-Tyrosine | 2500 | 500 | 10000 | mg |
| Pine Bark Extract | 200 | 75 | 600 | mg |
| N-Acetyl-L-Carnitine | 3000 | 600 | 4000 | mg |
| DL- Choline Bitartrate (100%) | 500 | 30 | 1000 | mg |
| Maca Root PE 0.6% | 500 | 500 | 3000 | mg |
| Glucosinolates | | | | |
| P5P (Pyridoxal-5-Phosphate) | 25 | 20 | 50 | mg |
| (100%) | | | | |
| Alpha Ketoglutaric Acid | 500 | 300 | 1000 | mg |

The present nootropic composition preferably provides cognitive stimulation, neurotransmitter and metabolic support, and an additional fuel source.

With specific reference to cognitive stimulation, the disclosed nootropic composition is preferably comprised of DL-Choline Bitartrate and theanine. Theanine is part of a group of substances referred to as methylxanthines, and DL-choline bitartrate is the salt for of choline, the precursor to acetylcholine. Together, DL-choline bitartrate and theanine provide a cognitive stimulant effect to the user. A worker skilled in the art would appreciate that theanine is an analogue of the amino acid L-glutamine and is found in particular plants and fungal species, including green tea. Theanine is also known to boost gamma-amino butyric acid (GABA), resulting in relaxation. Meanwhile, DL-choline bitartrate supports memory, improves cognitive performance and stimulates the mind by supporting the formation of the neurotransmitter acetylcholine. Together, DL-choline bitartrate and theanine in the present nootropic composition result in a wakeful state, improving cognitive performance.

With specific reference to neurotransmitter support, the present nootropic composition specifically provides support to neurotransmitters involved in enhancing cognition during times of stress. Neurons rely on highly specialized chemicals called neurotransmitters, which are chemical messengers that allow the transmission of signals from one neuron to the next. This in turn can affect every cell, tissue and system in your body. Dopamine functions as a neurotransmitter in the brain. As a precursor to epinephrine and norepinephrine, dopamine can initiate epinephrine signalling during the activation of the stress response in certain cells. The catecholamines, epinephrine and norepinephrine, are released as part of the fight-or-flight response, which is a physiological reaction in response to a perceived harmful event or threat to survival. The present nootropic composition uses tyrosine, which is converted to dihydroxyphenylalaine (L-DOPA), an amino acid precursor to the catecholamine neurotransmitters: dopamine, norepinephrine and epinephrine. The presence of tyrosine will enhance cognition and alertness and initiate a fight-or-flight response type of state. The present nootropic composition also is comprised of acetyl L-carnitine, one of several forms of L-carnitine supplements that are shown to offer major physical and mental benefits. These benefits include, but are not limited to, reducing mental fatigue, enhancing cognitive function and improving learning. Research indicates that acetyl L-carnitine also promotes the production of the neurotransmitter acetylcholine, which is associated with memory and learning. Acetyl L-carnitine also has potent antioxidant properties and can prevent and repair damage caused by free radicals in brain cells. The present nootropic composition is further comprised of pine bark extract, which preferably improves cognitive function and oxidative stress by increasing nitric oxide (NO) production. An increase in NO leads to increase in blood flow and oxygen supply to muscles. NO can also act as a neurotransmitter and can evoke the release of acetylcholine and catecholamines. It is suggested that the combination of tyrosine, pine bark extract and acetyl L-carnitine results in a synergistic effect to enhance and support neurotransmitter function, particularly the catecholamines, thus resulting in cognitive enhancement.

With specific reference to fuel source, the present nootropic composition is preferably comprised of the three naturally occurring branched chain amino acids: leucine, isoleucine and valine. These amino acids help reduce the harmful effect of stress on one's body and also provide additional energy. Additionally, both taurine and maca root reduce anxiety, while promoting relaxation and improving exercise performance, respectively. Research suggests that the addition of magnesium may benefit individuals by reducing anxiety. Magnesium is shown to bind and stimulate GABA receptors, restricting the release of stress hormones and acts ting as filter to preventing them from entering the brain. The magnesium, potassium and salt are also a source of electrolytes allowing the cells to generate energy.

With specific reference to metabolic support, the present nootropic composition is preferably comprised alpha ketoglutaric (AKG) acid, one of two ketone derivatives of glutaric acid. AKG acid is the rate limiting intermediate for the Krebs cycle, known to release stored energy. AKG acid can be synthesized from transamination of glutamate and oxaloacetate. A transamination reaction, performed by enzymes, involves the transfer of an amino group from an amino acid to an amino-group acceptor (such as oxaloacetate) to form -keto acid and a new amino acid. It is important to note that AKG acid can go through a transaminase reaction with an amino acid to form an a-keto acid and glutamate. The present nootropic composition is also preferably comprised of pyridoxal-5-phosphate (P5P), the active form of vitamin B6. P5P acts as a coenzyme in all transamination reactions and is included in the nootropic composition to aid in metabolism of amino acids and increase energy. Providing the active form ensures that persons with impaired liver function, celiac disease, and older adults can benefit from vitamin B6. The addition of P5P in the nootropic composition ensures that the transaminase reaction can take place to convert any excess glutamate to AKG acid, thereby allowing it to feed into the Krebs cycle.

The disclosed supplement provides different avenues to help increase energy and provide cognitive stimulation. As disclosed, DL-choline bitartrate and theanine work together by activating neurotransmitters important in relaxation, memory and attention. Furthermore, through tyrosine, acetyl L-carnitine and pine bark extract, the supplement provides additional neurotransmitter support involved in enhancing cognition and alertness. The supplement provides electrolytes as a resource for alternative energy and also provides the metabolic support through AKG and P5P, which increase energy and promote wakefulness.

In another embodiment, the present disclosure describes another nootropic composition. The nootropic composition is preferably comprised of at least L-theanine, L-tyrosine, pyridoxal-5-phosphate (P5P), pine bark extract, N-acetyl-L-carnitine, alpha ketoglutaric acid, medium-chain triglyceride (MCT) oil powder, betahydroxy butyrate and caffeine. The chart below illustrates the preferred daily dose and the preferred range of each ingredient.

TABLE D

| | Daily | Min | Max | |
|---|---|---|---|---|
| Ingredient | Dose | Range | Range | UoM |
| L-Theanine | 175 | 130 | 350 | mg |
| L-Tyrosine | 700 | 500 | 2000 | mg |
| P5P (Pyridoxal-5-Phosphate) (100%) | 25 | 20 | 50 | mg |
| Pine Bark Extract | 200 | 75 | 600 | mg |
| N-Acetyl-L-Carnitine | 1000 | 600 | 2000 | mg |
| Alpha Ketoglutaric Acid | 500 | 300 | 1000 | mg |
| MCT Oil Powder (50%) | 300 | 150 | 800 | mg |
| Betahydroxy Butyrate (magnesium salt) | 500 | 450 | 1000 | mg |
| Caffeine | 140 | 50 | 150 | mg |

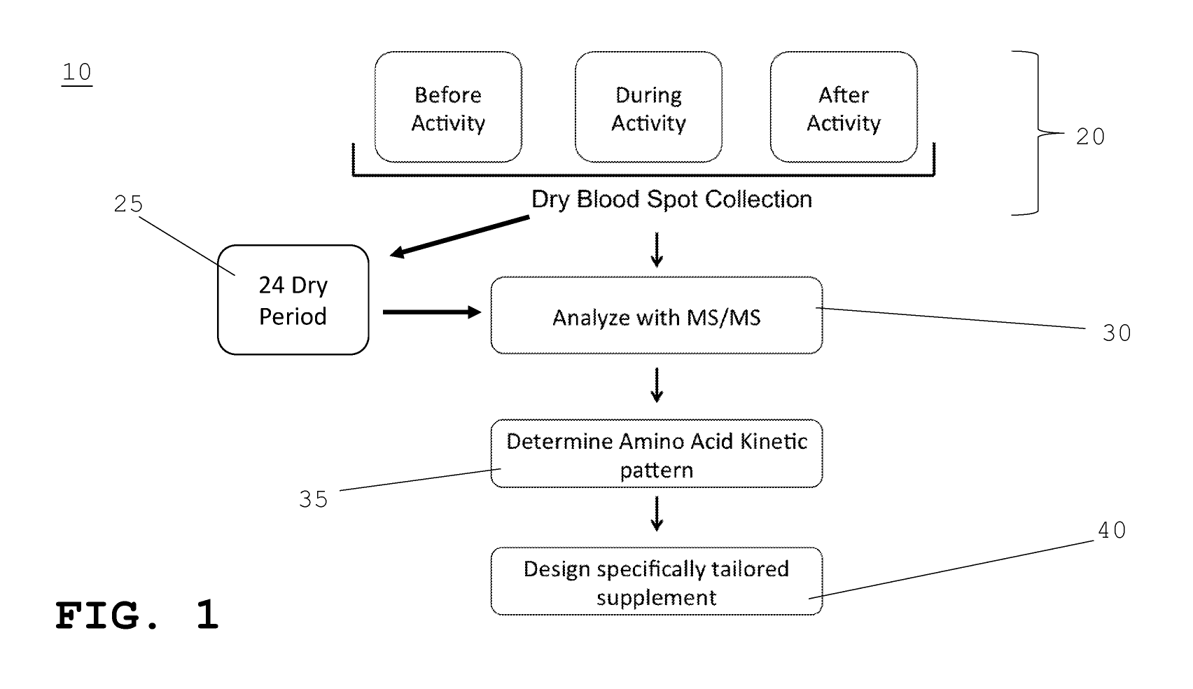

With reference to FIG. 1 and according to an embodiment of the present disclosure, the flow chart of a method 10 to design a specifically tailored supplement is shown. In a first step 20, dry blood spots (DBS) are collected before, during and after activity and preferably taken from fingertip skin puncture. The DBS can be collected from participants before, during and after exertion or stressor in either a fasted or unfasted state and stored on filter paper cards. In a second optional step 25, the samples are dried for 24 hours at room temperature. The filter paper cards containing DBS can be kept at room temperature and stored in the laboratory until analysis in a third step 30. In the third step 30, extracts of the DBS are analyzed with tandem mass spectrometry (MS/MS). Indeed, the metabolites that are collected on the DBS are extracted with polar organic solvents such as methanol and analyzed directly by mass spectrometry or via liquid chromatography (LC) MS/MS. For amino acid extraction, DBS on the cards are punched into a small circle with a hole-puncher and transferred into a tube containing methanol. The compounds extracted from this process, which include amino acid metabolites, are then run on an LC column prior to infusion preferably into a API 2000 triple quadrupole mass spectrometer using a Dionex™ Ultimate 3000 high performance liquid chromatograph. Each metabolite is ionized by electrospray ionization and quantified using multiple reaction monitoring experiments against the parent mass and two of the most abundant fragments. Metabolite quantities are normalized against an internal standard; typically, an isotopically labelled amino acid. A worker skilled in the art will appreciate that automation of the quantitation data can be achieved using software. In a fourth step 35, the amino acid kinetic pattern is determined. In a preferred embodiment, a volcano plot may first be utilized to display the significance versus the fold change. The maximum fold changes (MFC) in amino acid concentration and P-values of statistical hypothesis testing serves as a score to rank putative amino acid candidates. A worker skilled in the art would appreciate that the volcano plot is used in literature as a method of choice to analyze and visualize significant changes; however, other types of plots can be used in the art. MFC is calculated by the difference between observed minimum and maximum concentration values of the amino acid, independent from the time they appeared. In other words, it does not matter how the MFC is calculated, provided that it is the difference between the observed minimum and maximum at a single discrete time. MFC is based on the median concentration values extracted from the interpolated concentration curves. Therefore, if the concentration of minimum index is smaller than the concentration of maximum index, then the MFC is equal to concentrated maximum divided by the concentrated minimum. Conversely, if the concentration of minimum index is greater than the concentration of maximum index, then the MFC is equal to the concentrated minimum divided by the concentrated maximum.

As shown below:

If concentration minimum index<concentration maximum index, then

$$MFC = \frac{concentrated maximum}{concentrated minimum}$$

Conversely if concentration minimum index>concentration maximum index, then

$$MFC = \frac{concentrated minimum}{concentrated maximum}$$

The P-values of statistical hypothesis testing will be calculated in a similar fashion.

Of the amino acids that demonstrate significant changes, the relative concentration for each amino acid versus course of time is plotted. The curves are characterized by polynomial fitting of 9^{th} degree to the median concentration values of the analyzed amino acids. The polynomial fitting is used to generate a kinetic signature for each amino acid. In a fifth step 40, the specifically tailored supplement is determined based on the kinetic signature determined in the fourth step 35. The supplement will aid the recovery from or the performance during a specific activity or stressor. To generate the supplement, amino acids are grouped according function within the body or specific importance to a metabolic system. Changes in amino acids will then be grouped by time point, the time point defined as: early response, mid response, late response, and delayed response. This information is used to determine the metabolic status of the individual and the supplement is formulated specific to these changes in amino acids. The supplement dosage is based on the magnitude of the change in the kinetic pattern.

Many modifications of the embodiments described herein as well as other embodiments may be evident to a person skilled in the art having the benefit of the teachings presented in the foregoing description and associated drawings. It is understood that these modifications and additional embodiments are captured within the scope of the contemplated disclosure which is not to be limited to the specific embodiment disclosed.

1. A nootropic composition comprising: ascorbic acid, taurine, elemental magnesium glycinate, L-theanine, L-tyrosine, pine bark extract, N-acetyl-L-carnitine, DL-choline bitartrate, maca root, glucosinolates, P5P (pyridoxal-5-phosphate) and alpha ketoglutaric acid.

2. The nootropic composition of claim 1 where the ascorbic acid is within a range of 200-1000 mg, the taurine is within a range of 500-3000 mg, the elemental magnesium glycinate is within a range of 5-150 mg, the L-theanine is within a range of 130-350 mg, the L-tyrosine is within a range of 500-100000 mg, the pine bark extract is within a range of 75-600 mg, the N-acetyl-L-carnitine is within a range of 600-4000 mg, the DL-choline bitartrate is within a range of 30-1000 mg, the maca root is within a range of 500-3000 mg, the P5P is within a range of 20-50 mg and the alpha ketoglutaric acid is within a range of 300-1000 mg.

3. A skin supplement composition comprising: hydrolyzed collagen, glucosamine sulfate, biotin, vitamin A palmitate, vitamin E, alpha lipoic acid, coenzyme Q₁₀, pine bark extract, L-selenomethionone, and niacinamide.

4. The skin supplement composition of claim 3 wherein the hydrolyzed collagen is within a range of 500-3000 mg, the glucosamine sulfate is within a range of 500-1500 mg, the biotin is within a range of 0.05-0.2 mg, the vitamin A palmitate is within a range of 200-1000 mcg RAE, the vitamin E is within a range of 50-500 mg α-tocopherol, the alpha lipoic acid is within a range of 50-400 mg, the coenzyme Q₁₀ is within a range of 30-100 mg, the pine bark extract is within a range of 75-300 mg, the L-selenomethionone is within a range of 50-150 mcg and the niacinamide is within a range of 15-150 mg.

5. A method to provide a specifically tailored supplement, the steps comprising:
collecting dry blood spot samples;
analyzing the dry blood spots with tandem mass spectrometry;
determining an amino acid kinetic pattern based on the analysis; and,
generating the specifically tailor supplement based on the pattern.

6. The method of claim 5 further comprising the step of drying the dry blood spot samples for a period of 24 hours at room temperature.

7. The method of claim 5 wherein the dry blood spot samples are collected before, during and after physical activity.
